(19) United States
(12) Patent Application Publication
(10) Pub. No.: US 2024/0398540 A1
(43) Pub. Date: Dec. 5, 2024

(54) SIZE ADJUSTABLE AORTIC ENDOGRAFT FENESTRATION DEVICE AND RETRIEVABLE VESSEL MARKER

(71) Applicant: The University of Toledo, Toledo, OH (US)

(72) Inventors: Ayman Eltayeb Elhag Ahmed, Toledo, OH (US); Mohamed Fikri Mohamed Osman, Toledo, OH (US)

(73) Assignee: The University of Toledo, Toledo, OH (US)

(21) Appl. No.: 18/688,151

(22) PCT Filed: Aug. 30, 2022
(86) PCT No.: PCT/US2022/041969

§ 371 Date: Feb. 29, 2024

Related U.S. Application Data
(60) Provisional application No. 63/239,438, filed on Sep. 1, 2021.

(51) Int. Cl. A61F 2/06; A61B 90/00

(52) U.S. Cl. CPC A61F 2/06; A61B 90/39; A61B 2090/3966; A61B 2090/3987; A61F 2002/068; A61F 2240/002

(57) ABSTRACT

A fenestration device for fenestrating an endograft material, a retrievable vessel marker, and methods of using the same are described. The fenestration device includes a needle-like tip with cutting edges configured to make a perforation in an endograft material and blades configured to enlarge the perforation. The retrievable vessel marker includes a wire with a helical portion having four radiopaque markers thereon and blades extendable from the needle-like tip configured to be heated by an external heat source to enlarge the perforation in the endograft material.

### RELATED APPLICATIONS

This application claims priority to U.S. Provisional Application No. 63/239,438 filed under 35 U.S.C. § 111 (b) on Sep. 1, 2021, the disclosure of which is incorporated herein by reference in its entirety for all purposes.

### STATEMENT REGARDING FEDERALLY SPONSORED RESEARCH

This invention was made with no government support. The government has no rights in this invention.

### BACKGROUND

Every year, about 200,000 people in the United States are diagnosed with an abdominal aortic aneurysm (AAA). In general, endovascular aortic aneurysm repair (EVAR) is the standard of care for AAA. However, about 20-30% of AAA patients are not eligible for standard EVAR. People who are not candidates for EVAR can be treated with open repair, which is a very high-risk procedure with elevated levels of perioperative complications that may not be compatible with some frail patients. The other option is treatment with a custom-made fenestrated or branched endovascular graft, which requires an expensive endograft that has to be requested about six weeks before placement, making this not a good option in urgent or emergent situations. It would be advantageous to provide new and improved devices and methods to treat such patients.

### SUMMARY

Provided is a fenestration device comprising a steerable sheath defining an inner lumen; a cutting member disposed in the inner lumen, wherein the cutting member has a distal end and a proximal end and is movable between a retracted position and an extended position, the distal end of the cutting member being disposed within the steerable sheath in the retracted position, and the distal end of the cutting member protruding from the steerable sheath in the extended position; a needle-like tip on the distal end of the cutting member, the needle-like tip having cutting edges; and blades movable between a first position and a second position, the blades being disposed within the needle-like tip in the first position, and the blades protruding from the needle-like tip in the second position.

In certain embodiments, the fenestration device further comprises a control knob on the steerable sheath configured to control a height of the blades.

In certain embodiments, the cutting member defines a bore configured to receive a guide wire. In particular embodiments, the fenestration device further comprises a guide wire disposed in the bore. In particular embodiments, the fenestration device further comprises a proximal opening at a proximal end of the cutting member, wherein the proximal opening is configured to receive a guide wire for insertion into the bore.

In certain embodiments, the blades have a height ranging from about 3 mm to about 5 mm, and a length of about 10 mm.

In certain embodiments, the blades comprise a thermally conductive material.

In certain embodiments, the fenestration device further comprises a port on the distal end of the cutting member configured to connect an electrocautery wire within the cutting member to a heat source. In particular embodiments, the port is connected to an electrocautery wire that extends through a bore in the cutting member to the blades.

In certain embodiments, the sharp cutting edges are configured to perforate an endograft material.

In certain embodiments, the fenestration device further comprises a control knob on the steerable sheath configured to control movement of the blades. In particular embodiments, the fenestration device further comprises a central gear and teeth within the needle-like tip, wherein the central gear is connected to the teeth; wherein each of the teeth connects the central gear to one of the blades; wherein rotation of the central gear in a first direction causes extension of the blades outward from a surface of the needle-like tip, and rotation of the central gear in a second direction causes retraction of the blades inward; and wherein rotation of the control knob causes rotation of the central gear.

Further provided is a method of fenestrating an endograft, the method comprising positioning the fenestration device described herein within an endograft near a branch vessel; extending the needle-like tip outward from the steerable sheath to create a perforation in the endograft with the sharp cutting edges; running a guide wire through a bore in the cutting member into the branch vessel; enlarging the perforation by extending the blades out from the needle-like tip; and withdrawing the fenestration device from the endograft, leaving the guide wire in the branch vessel.

In certain embodiments, the extending comprises moving the cutting member to the second position by pushing the cutting member distally through the inner lumen of the steerable sheath.

In certain embodiments, the method further comprises applying heat to the blades while enlarging the perforation. In certain embodiments, the branch vessel is a renal artery.

In certain embodiments, the method further comprises shunting the endograft into the branch vessel.

In certain embodiments, the method further comprises placing a catheter over the guide wire in the branch vessel; retracting the guide wire through the catheter, leaving the catheter in the branch vessel; inserting a retrievable vessel marker into the catheter, wherein the retrievable vessel marker comprises a wire having four radiopaque markers on a helical portion of the wire at a distal end of the wire; and removing the catheter, leaving the retrievable vessel marker in the branch vessel. In particular embodiments, the method further comprises rotating the retrievable vessel marker under fluoroscopy to orient the radiopaque markers to mark desired quadrants of the branch vessel.

Further provided is a retrievable vessel marker comprising a catheter defining a lumen; and a wire removable from the catheter and movable from a first position and a second position, the wire disposed within the catheter in the first position, and the wire protruding from the catheter in the second position; wherein the wire includes a helical portion at a distal end of the wire, the helical portion protruding from the catheter in the second position, and the wire comprises four radiopaque markers on the helical portion.

Further provided is a retrievable vessel marker comprising a catheter defining a lumen; and a wire removable from the catheter and movable from a first position wherein the wire is disposed within the catheter to a second position wherein the wire protrudes from the catheter; wherein the wire includes a helical portion at a distal end, the helical portion protruding from the catheter in the second position, and the wire comprises four radiopaque markers on the helical portion.

Further provided is a method for marking a position or orientation of a blood vessel, the method comprising advancing a catheter over a guide wire disposed in a blood vessel; removing the guide wire from the catheter to leave the catheter positioned within the blood vessel; advancing a wire through the catheter, wherein the wire has a helical portion at a distal end, the helical portion comprising four radiopaque markers thereon; and retracting the catheter to leave the distal end with the radiopaque markers in the blood vessel. In certain embodiments, the method comprises rotating the wire to mark quadrants of the blood vessel with the radiopaque markers.

Further provided is a fenestration device comprising a needle-like tip configured to create a perforation in an endograft material; and blades extendable from the needle-like tip configured to be heated by an external heat source to enlarge the perforation in the endograft material.

### BRIEF DESCRIPTION OF THE DRAWINGS

The patent or application file may contain one or more drawings executed in color and/or one or more photographs.

FIG. 1: Perspective view of a non-limiting example embodiment of a fenestration device.

FIG. 2: Perspective view of a non-limiting example embodiment of a fenestration device with the needle-like tip protruding from the steerable sheath.

FIG. 3: Perspective views of a non-limiting embodiment of a fenestration device with the needle-like tip protruding from the steerable sheath and the blades protruding from the needle-like tip.

FIG. 4: Cross-sectional illustration of the needle-like tip of a fenestration device showing the blades being movable by a central gear and teeth.

FIGS. 5A-5E: Illustration of a non-limiting example procedure for using a fenestration device.

FIG. 6: Perspective view of a retrievable vessel marker in an after-deployment configuration.

FIG. 7: Perspective view of a retrievable vessel marker in a before-deployment configuration.

FIGS. 8A-8E: Illustration of a non-limiting example procedure for using a retrievable vessel marker.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Provided herein is a fenestration device configured for fenestrating an endograft. The fenestration device is particularly useful in certain abdominal aortic aneurysm (AAA) treatments, but is by no means limited to use in treating an AAA. Treating a paravisceral AAA or thoracoabdominal aortic aneurysm presents a challenge because the aneurysm involves major branches. Conventionally, lasers have been used to make holes in grafts to add stent grafts to accommodate the branches. However, the laser technique is not FDA approved, and is expensive, making it not a practical option for many patients. Advantageously, the fenestration device provided herein is capable of making holes in a graft material with a needle or sharp cutting edges, thereby eliminating the need for a laser to fenestrate the graft material.

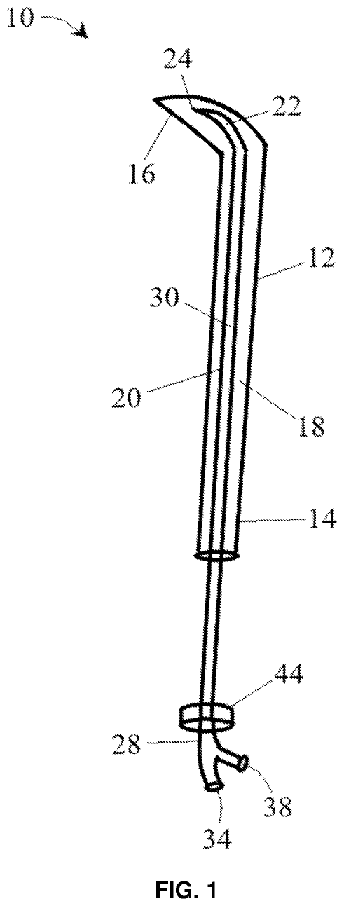

Referring now to FIGS. 1-5, depicted is a fenestration device 10. The fenestration device 10 includes a steerable sheath 12 having a proximal end 14 and a distal end 16 and defining an inner lumen, also referred to as an inner core 18. The steerable sheath 12 has a size, and is made of a material, configured to allow the steerable sheath 12 to be inserted into a blood vessel 41 or endograft 40. In the illustrated example, the steerable sheath 12 is substantially L-shaped. In some embodiments, the steerable sheath 12 is configured to be bent to be substantially L-shaped. However, it should be appreciated that in other embodiments, the steerable sheath 12 can be shaped differently to allow the steerable sheath 12 to be inserted into a blood vessel 41 or endograft 40. Furthermore, the term “steerable” refers to the fact that the distal end 16 of the steerable sheath 12 can be rotated or bent.

Referring still to FIGS. 1-5, the inner lumen 18 houses a cutting member 20 defining a bore 30 and having a needle-like tip 22 at a distal end 24 and a control knob 44 at a proximal end 28. The cutting member 20 is made of an elongated, rigid material that is both rotatable within the inner lumen 18 and movable in a distal direction or proximal direction (i.e., toward the proximal end 14 or toward the distal end 16 of the steerable sheath 12) to cause the needle-like tip 22 to protrude from the inner lumen 18 or retract back into the inner lumen 18, respectively. The cutting member 20 can be moved distally or proximally within the inner lumen 18 by manually pushing or pulling on the proximal end 28 of the cutting member 20, respectively. The steerable sheath 12 includes an expandable opening 13 at the distal end 16 configured to allow the needle-like tip 22 to protrude out from the steerable sheath 12.

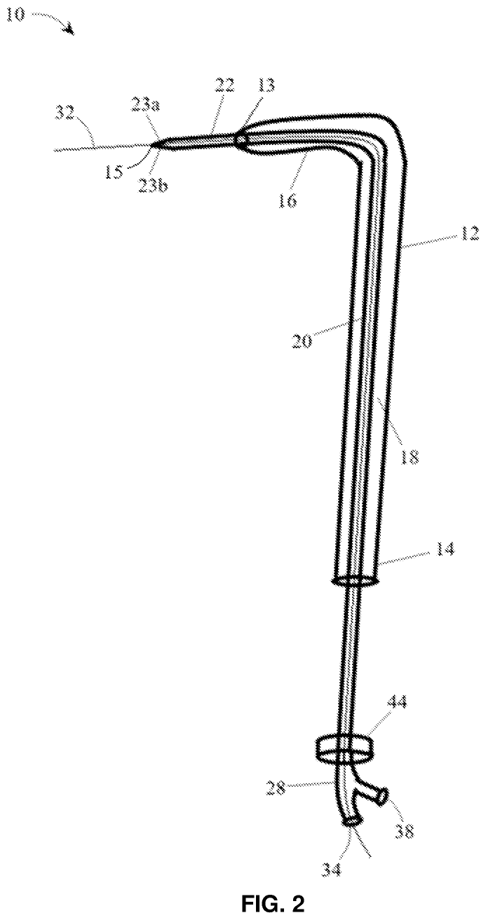

Referring now to FIG. 2, the needle-like tip 22 includes sharp cutting edges 23a, 23b. The sharp cutting edges 23a, 23b are configured to perforate endograft material. The sharp cutting edges 23a. 23b may be made from any hard material suitable for cutting endograft material, such as, but not limited to, stainless steel.

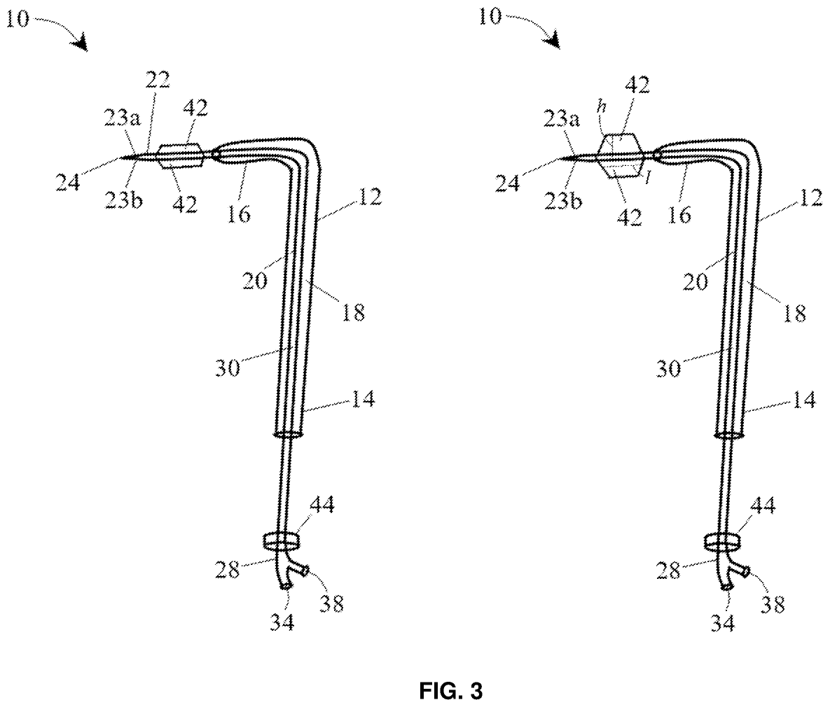

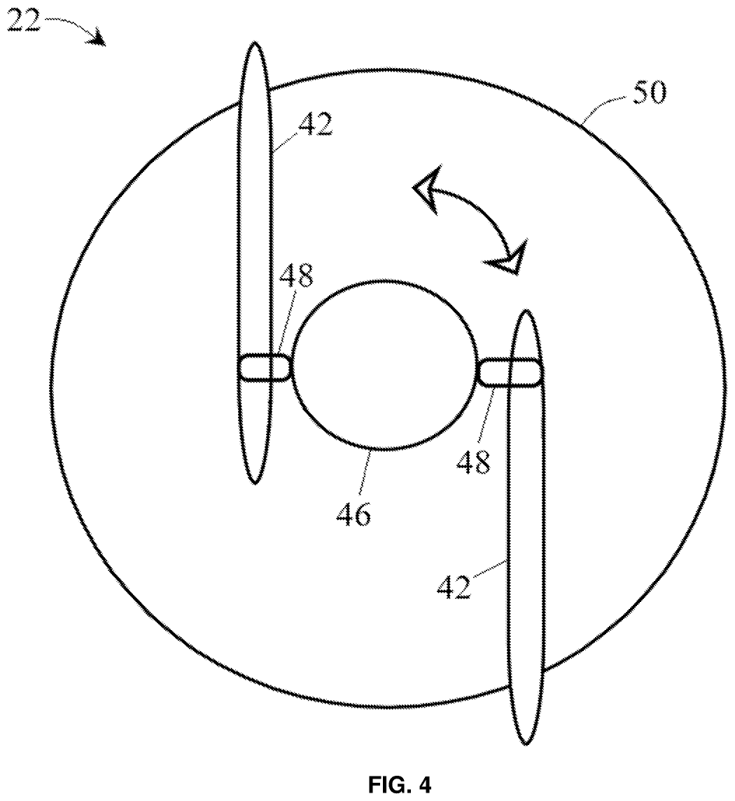

Referring now to FIG. 3, the needle-like tip 22 includes two or more blades 42 that are extendable from, and retractable within, the needle-like tip 22. Though the blades 42 may be referred to as cutting blades because they may be used to enlarge a perforation in the endograft material through mechanical cutting, the blades 42 do not actually need to be sharp. Rather, the blades 42 can be merely surfaces that can transfer heat conveyed to the blades 42 by electrocautery wires 36. The use of heat to cut endograft material can be preferable over mechanical cutting because it can result in more controlled cutting of the endograft material. Accordingly, the blades 42 do not need to be a sharp material, and can instead be any thermally conductive material such as, but not limited to, a metal or ceramic. For ease of illustration, FIG. 4 depicts the blades 42 as having pointy ends for cutting; however, this is not necessary. The blades 42 can have any shape that allows for the blades 42 to (i) be extended out from the needle-like tip 22, (ii) be retracted back into the needle-like tip 22, and (iii) enlarge a perforation in an endograft material by conducting heat to the endograft material and/or physically cutting the endograft material.

Referring to FIGS. 1-5, the extension and retraction of the blades 42 from the needle-like tip 22 can be performed by rotating the control knob 44 at the proximal end 28 of the cutting member 20. In other words, the control knob 44 may control the height h of the blades 42. Referring now to FIG. 4, which depicts a non-limiting example of a cross-sectional view from within the needle-like tip 22, a central gear 46 may be rotated by rotating the control knob 44. The central gear 46 is connected to teeth 48 on which are mounted the blades 42. The number of teeth 48 on the central gear 46 corresponds to the number of blades 42. In FIG. 4, the fenestration device 10 is shown with two blades 42 and two teeth 48 on the central gear 46. However, the fenestration device 10 is not limited to two blades 42 and two teeth 48. Rather, in some embodiments, the fenestration device 10 includes four blades 42 and four teeth 48. In some embodiments, the fenestration device 10 includes six blades 42 and six teeth 48. In some embodiments, the fenestration device 10 includes one cutting blade 42 and one tooth 48.

Referring still to FIG. 4, the central gear 46 is turnable to cause the blades 42 to protrude from, or retract into, the needle-like tip 22. As the central gear 46 is rotated in the directions depicted by the double-sided arrow in FIG. 4, the teeth 48 are correspondingly moved, which causes actuation of the blades 42 out of a surface 50 of the needle-like tip 22. In the embodiment depicted in FIG. 4, which shows the blades 42 partially protruding out of the needle-like tip 22, clockwise rotation of the central gear 46 causes the blades 42 to further protrude out of the needle-like tip 22, and counterclockwise rotation of the central gear 46 causes the blades 42 to retract within the needle-like tip 22. The central gear 46 may be connected to the control knob 44 through a rotatable central member housed within the bore 30, which is not depicted for ease of illustration. However, it is understood that the central gear 46 may be connected to the control knob 44 through any possible apparatus to cause rotation of the central gear 46 upon rotation of the control knob 44. Furthermore, other mechanisms of causing the extension and retraction of the blades 42 are possible and entirely encompassed within the scope of the present disclosure. For example, the control knob 44 does not need to be a knob. Rather, the control knob 44 can be any physical structure capable of controlling the extension and retraction of the blades 42. As another example, instead of utilizing a central gear 46 and teeth 48 to cause the extension and retraction of the blades 42, the needle-like tip 22 can include a double-sided screw with a central jack connected to the control knob 44 such that rotation of the control knob 44 in a first direction causes the double-sided screw to push the blades 42 out of the needle-like tip 22, and rotation of the control knob 44 in a second direction causes the double-sided screw to retract the blades 42 back into the needle-like tip 22.

Referring now to FIG. 3, the blades 42 may have a length/of about 10 mm, and a height h that ranges from about 3 mm to about 5 mm when extended. However, other dimensions of the blades 42 are possible and encompassed within the scope of the present disclosure.

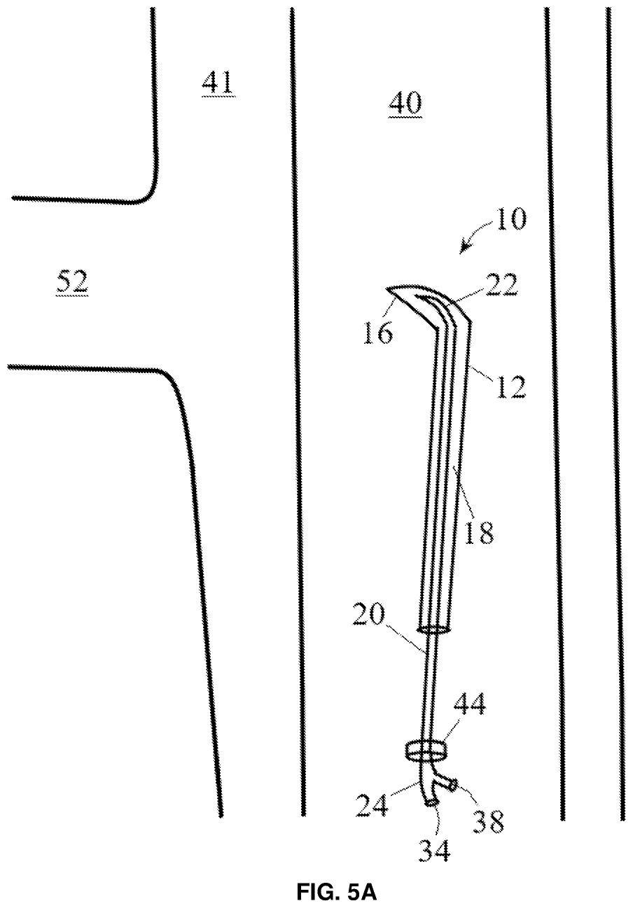

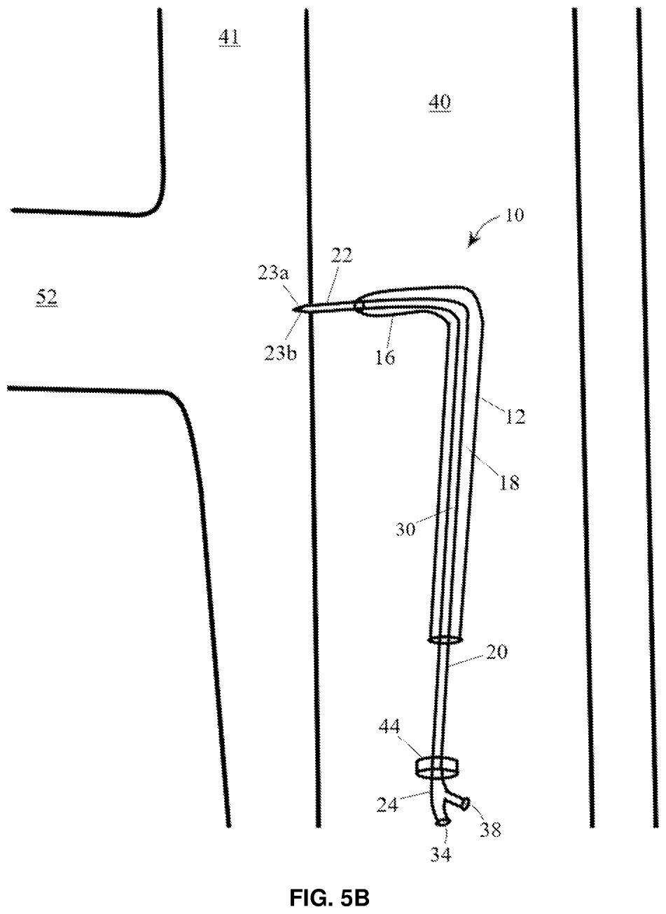

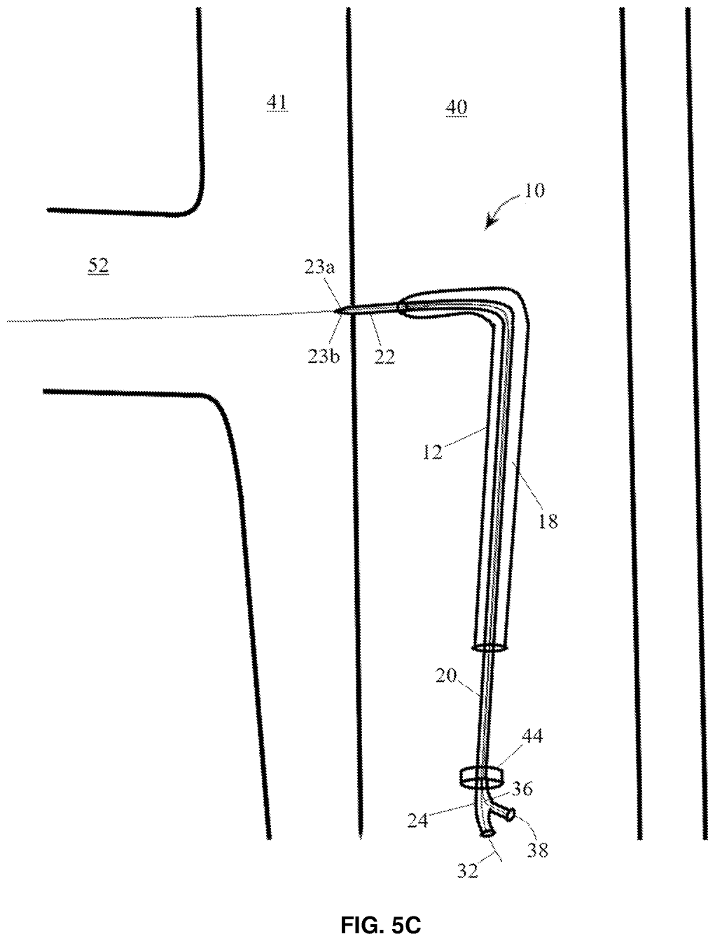

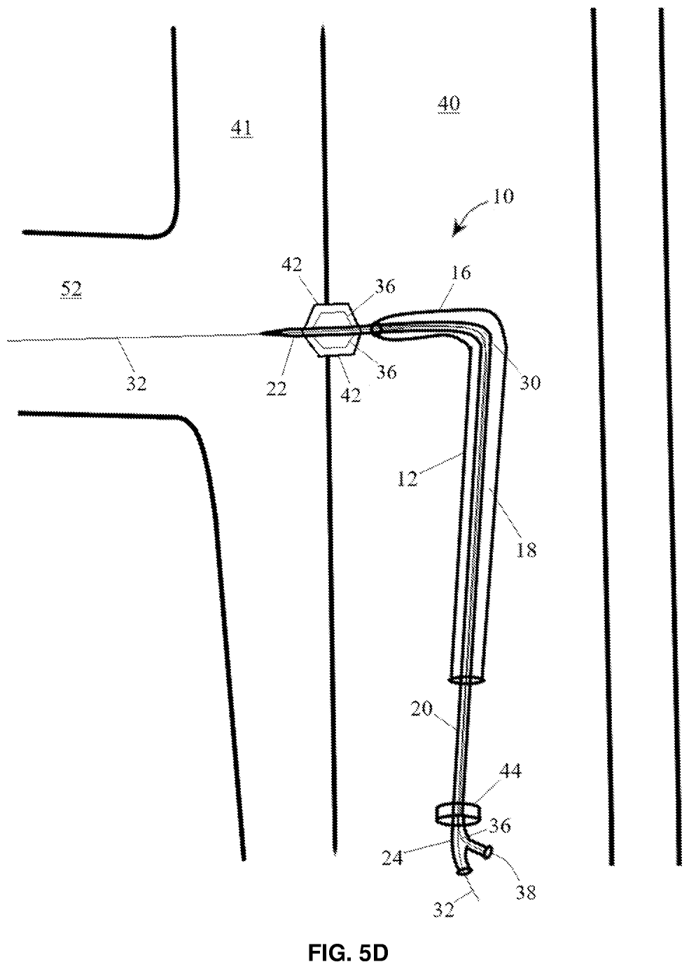

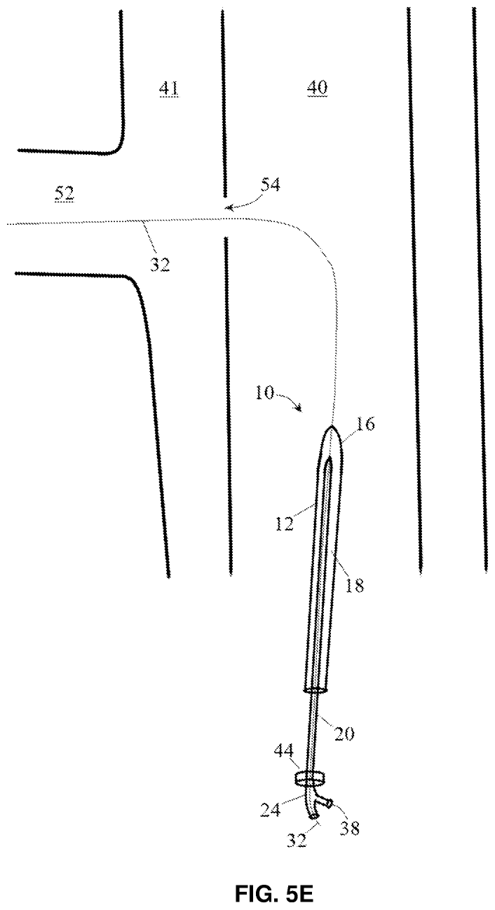

Referring to FIGS. 1-5, but as best seen in FIG. 5C, the bore 30 of the cutting member 20 is configured to receive a guide wire 32. The needle-like tip 22 includes an opening 15 configured for the guide wire 32 to pass through. At the proximal end 28, the cutting member 20 includes a proximal opening 34 configured for the guide wire 32 to be inserted into the cutting member 20. The guide wire 32 can be inserted through the proximal opening 34 and pushed through the inner lumen 18 to the distal end 24 of the cutting member 20, where the guide wire 32 may be pushed to protrude through the opening 15 of the needle-like tip 22. As shown in FIG. 5C, the guide wire 32 may be pushed into a branch vessel 52 to facilitate the placement of a shunt graft following fenestration with the fenestration device 10.

Referring now to FIGS. 5C-5D, the bore 30 of the cutting member 20 may also house electrocautery wires 36 configured to supply heat to the blades 42 provided by an electrocautery machine attached to a port 38 at the proximal end 28 of the cutting member 20. A suitable electrocautery machine. external to the fenestration device 10, may be connected to the port 38 to provide a source of heat to the electrocautery wires 36. The electrocautery wires 36 may run from the port 38 through the inner lumen 18 to the needle-like tip 22 where the electrocautery wires 36 provide heat to the blades 42. In such embodiments, the blades 42 are made of a thermally conductive material, and the electrocautery wires 36 may simply be in contact with the blades 42 to transfer heat to the blades 42.

Referring now to FIGS. 5A-SE, depicted is a non-limiting example procedure for using a fenestration device 10 to fenestrate an endograft 40. First, the endograft 40 is placed in the vessel 41, or is otherwise already positioned inside the vessel 41. As a non-limiting example, the endograft 40 may be an aortic endograft and the vessel 41 may be the aorta. As depicted in FIG. 5A, the fenestration device 10 is positioned within an endograft 40 inside the vessel 41 near a branch vessel 52 such as, but not limited to, the renal artery. To do this, a wire may be placed over which the fenestration device 10 is passed, and the wire can then be retrieved. Alternatively, the fenestration device 10 may be guided into the endograft 40 without passing the fenestration device 10 over a wire. Once positioned inside the endograft 40, the steerable sheath 12 can be bent under fluoroscopy guidance to face the branch vessel 52 of interest. As depicted in FIG. 5B, the needle-like tip 22 is extended outward from the steerable sheath 12 by pushing the proximal end 28 of the cutting member 20 in the distal direction, and the sharp cutting edges 23a, 23b of the needle-like tip 22 perforate the endograft 40 in the desired location adjacent to the branch vessel 52. The needle-like tip 22 is thus advanced to pierce through the fabric of the endograft 40. As depicted in FIG. 5C, the guide wire 32 may be passed through the bore 30 once the endograft 40 has been perforated, such that the guide wire 32 is inserted into the branch vessel 52. Then, as depicted in FIG. 5D, the perforation in the endograft 40 can be enlarged by extending the blades 42 out from the needle-like tip 22 to the desired height h (for example, 3-5 mm) using the control knob 44. Heat may be applied to the blades 42 through the electrocautery wires 36 (which may extend across the blades 42) during this step, though this is not strictly necessary. Advantageously, the application of heat allows for the enlargement of the perforation 54 to be performed in a controlled manner. Regardless of whether heat is used, once the perforation 54 is enlarged to the desired extent, the blades 42 may then be retracted back into the needle-like tip 22 using the control knob 44. As depicted in FIG. 5E, the fenestration device 10 may then be retrieved over the guide wire 32 and withdrawn from the endograft 40, leaving the guide wire 32 in the perforation 54 and the branch vessel 52. At this point, the endograft 40 is ready for shunting into the branch vessel 52. A stent graft can be placed to bridge the endograft 40 to the branch vessel 52.

Though the fenestration device 10 is not limited to use in treating AAA, the fenestration device 10 may facilitate treatment of a paravisceral AAA or thoracoabdominal aortic aneurysm using an endovascular technique while at the same time creating fenestrations that allow the passage of bridging stents to graft side branches in order to maintain the flow to the branch vessels from the aorta, and also exclude the flow from the aortic aneurysm. The fenestration device 10 may also be used to retrofit a graft in vivo, for example if it is determined that a previously placed graft is obstructing blood flow to or from a branch vessel 52. The fenestration device 10 may be used to fenestrate vessels in cases of inadvertently or intentionally covered branch vessels 52.

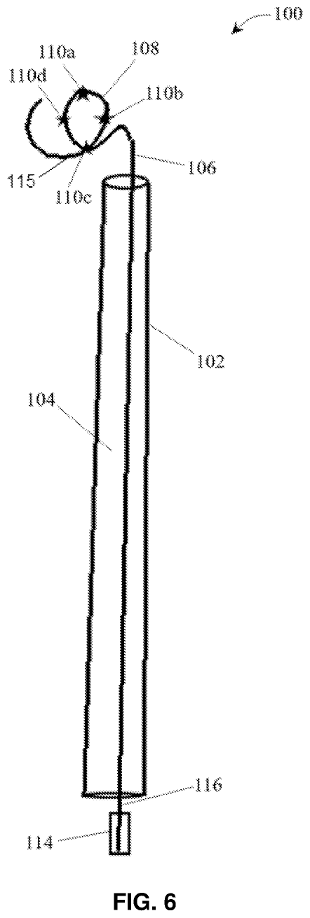

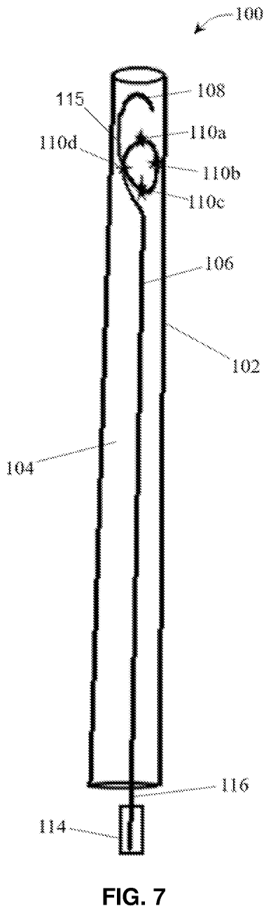

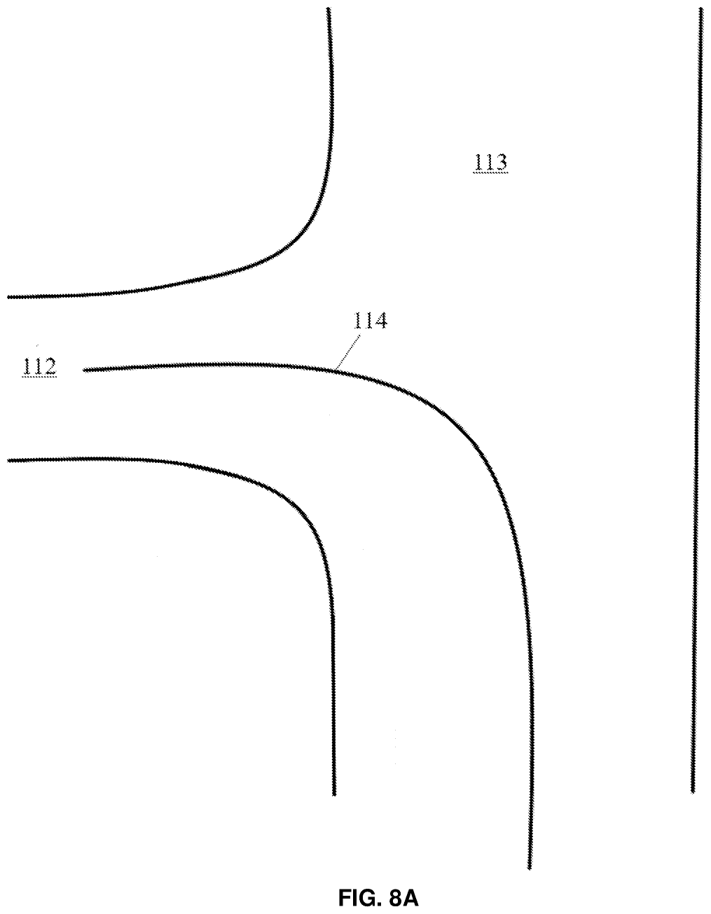

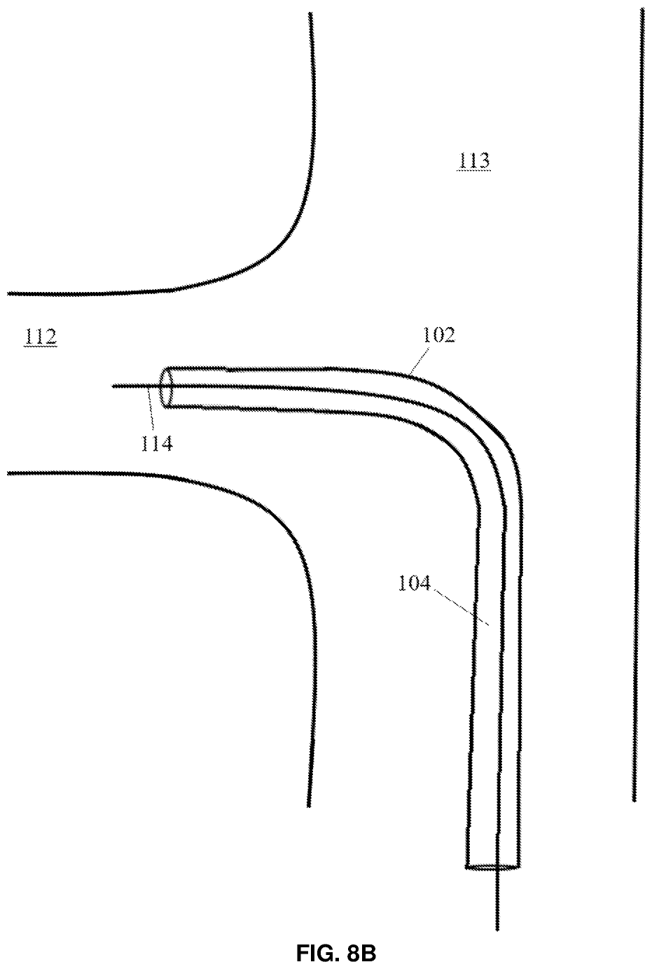

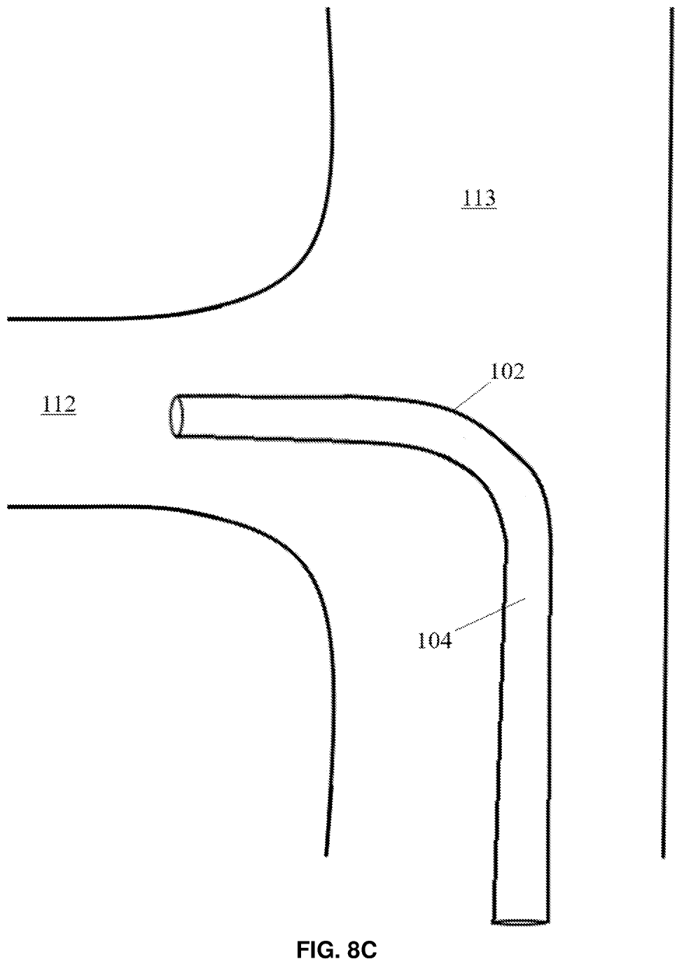

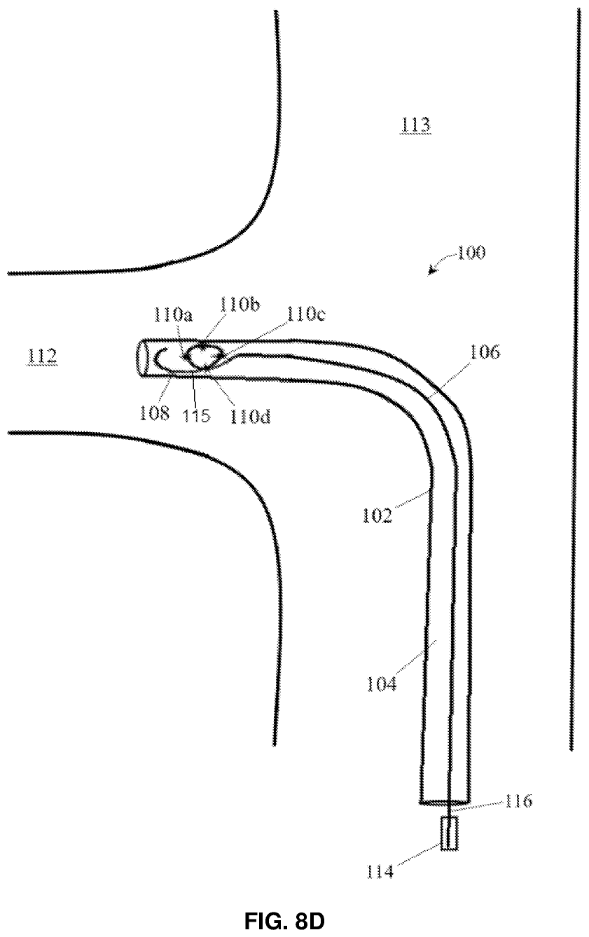

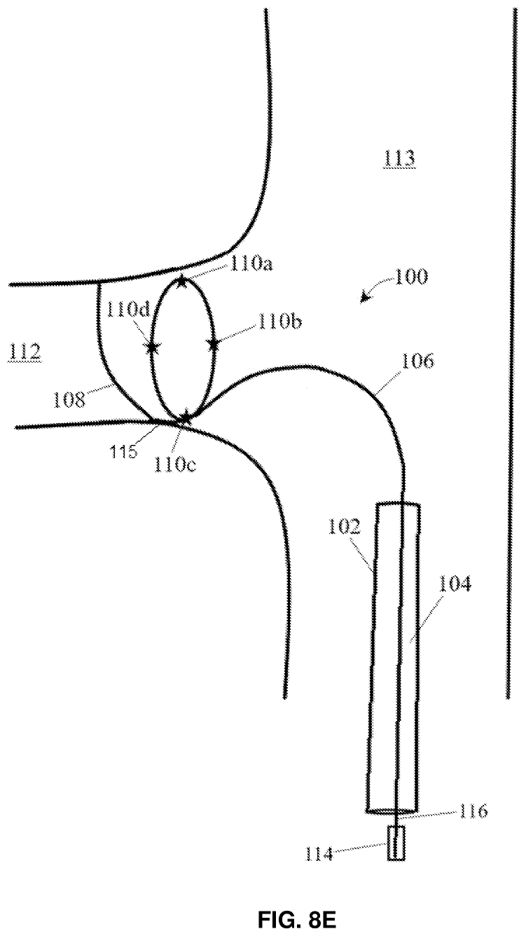

Referring now to FIGS. 6-8, depicted is a retrievable vessel marker 100. FIG. 6 depicts the retrievable vessel marker 100 in a post-deployment configuration, and FIG. 7 depicts the retrievable vessel marker 100 in a pre-deployment configuration. The retrievable vessel marker 100 may be used in conjunction with the fenestration device 10, but may also be used independently of the fenestration device 10, to mark a vessel location and/or orientation. The retrievable vessel marker 100 may include a catheter 102 defining a lumen 104, and a wire 106 movable within the catheter 102 from a first position in which the wire 106 is disposed within the lumen 104 to a second position in which the wire 106 protrudes from the catheter 102. The wire 106 includes a helical portion 108 at a distal portion 115, where the helical portion 108 protrudes from the catheter 102 in the second position. The wire 106 includes four radiopaque markers 110a, 110b, 110c. 110d on the helical portion 108, configured to illustrate the geography of a vessel in which the helical portion 108 is disposed. In other words, the four radiopaque markers 110a-110d may be disposed at the north, south, east, and west sides of a vessel, as depicted in FIG. 8E. Notably, the retrievable vessel marker 100 does not need to include the catheter 102, as the catheter 102 is a deployment vehicle for the wire 106 having the radiopaque markers 110a-110d thereon, but not itself a marker. The wire 106 is removable in its entirety from the catheter 102.

Referring still to FIGS. 6-8, the retrievable vessel marker 100 may include a handle 114 at a proximal end 116 of the wire 106 for a user to hold. The handle 114 may be used to rotate the wire 106, and thereby rotate the helical portion 108 to orient the radiopaque markers 110a-110d into a proper position under fluoroscopy. The handle 114 may also be used to push or pull the wire 106 into or out of the catheter 102.

Referring now to FIGS. 8A-8E, depicted is a non-limiting example procedure for using a retrievable vessel marker 100 to mark a branch vessel 112. As depicted in FIG. 8A, a guide wire 114 may be present in the branch vessel 112 of interest. As depicted in FIG. 8B, the catheter 102 may be advanced over the guide wire 114 through a vessel 113 to the branch vessel 112. The guide wire 114 may then be removed from the catheter 102 to leave the catheter 102 positioned within the branch vessel 112, as depicted in FIG. 8C. Then, the wire 106 with the helical portion 108 may be advanced through the catheter 102, as depicted in FIG. 8D. Finally, as depicted in FIG. 8E, the retrievable vessel marker 100 may be deployed by retracting the catheter 102, effectively marking the geography of the branch vessel 112 with the radiopaque markers 110a-110d. Optionally, the retrievable vessel marker 100 may later be removed through any suitable procedure.

As noted above, the retrievable vessel marker 100 may be used in conjunction with the fenestration device 10. For example, the fenestration device 10 may be used to fenestrate an endograft 40 near a branch vessel 52, into which the guide wire 114 may be placed. The guide wire 114 may then be retracted into a catheter 102, the wire 106 can be inserted into the catheter 102, and the catheter 102 can subsequently be withdrawn to leave the radiopaque markers 110a-110d in the branch vessel 52 to mark the branch vessel 52 for the placement of a shunt graft. In other embodiments, the retrievable vessel marker 100 may be employed to mark a branch vessel 52 prior to using the fenestration device 10 in order to fenestrate an endograft 40 nearby the branch vessel 52. However, the retrievable vessel marker 100 is by no means limited to use in connection with the fenestration device 10.

Certain embodiments of the devices and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the devices and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

1. A fenestration device comprising:
a steerable sheath defining an inner lumen;
a cutting member disposed in the inner lumen, wherein the cutting member has a distal end and a proximal end and is movable between a retracted position and an extended position, the distal end of the cutting member being disposed within the steerable sheath in the retracted position, and the distal end of the cutting member protruding from the steerable sheath in the extended position;
a needle-like tip on the distal end, the needle-like tip having cutting edges; and
blades movable between a first position and a second position, the blades being disposed within the needle-like tip in the first position, and the blades protruding from the needle-like tip in the second position.

2. The fenestration device of claim 1, further comprising a control knob on the steerable sheath configured to control a height of the blades.

3. The fenestration device of claim 1, wherein the cutting member defines a bore configured to receive a guide wire.

4. The fenestration device of claim 3, further comprising a guide wire disposed in the bore.

5. The fenestration device of claim 3, further comprising a proximal opening at a proximal end of the cutting member, wherein the proximal opening is configured to receive a guide wire for insertion into the bore.

6. The fenestration device of claim 1, wherein the blades comprise a thermally conductive material.

7. The fenestration device of claim 1, further comprising a port on the distal end of the cutting member configured to connect an electrocautery wire within the cutting member to a heat source.

8. The fenestration device of claim 7, wherein the port is connected to an electrocautery wire that extends through a bore in the cutting member to the blades.

9. The fenestration device of claim 1, further comprising a control knob on the steerable sheath configured to control movement of the blades.

10. The fenestration device of claim 9, further comprising a central gear and teeth within the needle-like tip;
wherein the central gear is connected to the teeth;
wherein each of the teeth connects the central gear to one of the blades;
wherein rotation of the central gear in a first direction causes extension of the blades outward from a surface of the needle-like tip, and rotation of the central gear in a second direction causes retraction of the blades inward; and
wherein rotation of the control knob causes rotation of the central gear.

11. (canceled)

12. A method of fenestrating an endograft, the method comprising:
positioning the fenestration device of claim 1 within an endograft near a branch vessel;
extending the needle-like tip outward from the steerable sheath to create a perforation in the endograft with the cutting edges;
running a guide wire through a bore in the cutting member into the branch vessel;
enlarging the perforation by extending the blades out from the needle-like tip into the second position; and
withdrawing the fenestration device from the endograft, leaving the guide wire in the branch vessel.

13. The method of claim 12, wherein the extending comprises moving the cutting member to the second position by pushing the cutting member distally through the inner lumen of the steerable sheath.

14. The method of claim 12, further comprising applying heat to the blades while enlarging the perforation.

15. The method of claim 12, wherein the branch vessel is a renal artery.

16. The method of claim 12, further comprising shunting the endograft into the branch vessel.

17. The method of claim 12, further comprising:
placing a catheter over the guide wire in the branch vessel;
retracting the guide wire through the catheter, leaving the catheter in the branch vessel;
inserting a retrievable vessel marker into the catheter, wherein the retrievable vessel marker comprises a wire having four radiopaque markers on a helical portion of the wire at a distal end of the wire; and
removing the catheter, leaving the retrievable vessel marker in the branch vessel.

18. The method of claim 17, further comprising rotating the retrievable vessel marker under fluoroscopy to orient the radiopaque markers to mark quadrants of the branch vessel.

19. A retrievable vessel marker comprising:
a catheter defining a lumen; and
a wire removable from the catheter and movable from a first position and a second position, the wire disposed within the catheter in the first position, and the wire protruding from the catheter in the second position;
wherein the wire includes a helical portion at a distal end of the wire, the helical portion protruding from the catheter in the second position, and the wire comprises four radiopaque markers on the helical portion.

20. A method for marking a position or orientation of a blood vessel, the method comprising:
advancing a catheter over a guide wire disposed in a blood vessel;
removing the guide wire from the catheter to leave the catheter positioned within the blood vessel;
advancing a wire through the catheter, wherein the wire has a helical portion at a distal end, the helical portion comprising four radiopaque markers thereon; and
retracting the catheter to leave the distal end with the radiopaque markers in the blood vessel.

21. The method of claim 20, comprising rotating the wire to mark quadrants of the blood vessel with the radiopaque markers.

22. A fenestration device comprising:
a needle-like tip configured to create a perforation in an endograft material; and
blades extendable from the needle-like tip configured to be heated by an external heat source to enlarge the perforation in the endograft material.
